(19) United States
(12) Patent Application Publication
(10) Pub. No.: US 2010/0145122 A1
(43) Pub. Date: Jun. 10, 2010

(54) Diisobutylene process

(76) Inventors: Thomas S. Zak, West Chester, PA (US); David W. Leyshon, West Chester, PA (US)

Correspondence Address: LyondellBasell Industries, 3801 WEST CHESTER PIKE, NEWTOWN SQUARE, PA, 19073, US

(21) Appl. No.: 12/315,605

(22) Filed: Dec. 4, 2008

(51) Int. Cl. C07C 2/06

(52) U.S. Cl. 585/506

(57) ABSTRACT

This invention is a process for producing diisobutylene from isobutylene. The process comprises first passing water through a downflow reactor containing a bed of sulfonic acid resin to produce an effluent stream having a pH of at least 5, then dimerizing isobutylene by contacting the sulfonic acid resin with a reaction feed comprising isobutylene and tertiary butyl alcohol. The downflow reactor comprises a bottom, an inlet located above the resin bed, an outlet located below the resin bed, and inert material in the space from the bottom of the reactor to at least above the outlet.

### FIELD OF THE INVENTION

This invention relates to a process for producing diisobutylene from isobutylene.

### BACKGROUND OF THE INVENTION

The dimerization of olefins such as isobutylene using a sulfonic acid-type ion exchange resin catalyst is well known in the art. For instance, U.S. Pat. No. 4,100,220 describes isobutylene dimerization using a sulfonic acid resin catalyst and tertiary butyl alcohol (TBA) as a selectivity enhancing modifier to produce diisobutylene (DIB). In addition, U.S. Pat. No. 4,447,668 discloses isobutylene dimerization using sulfonic acid resin catalyst A-15 with methyl t-butyl ether as solvent. U.S. Pat. No. 5,877,372 describes the selective dimerization of isobutylene using a sulfonic acid resin catalyst, TBA selectivity enhancing modifier and isooctane diluent. U.S. Pat. No. 6,376,731 further discloses the dimerization of isobutylene in the presence of a C₃-C₄ alkane diluent to enhance dimerization selectivity and TBA to promote selectivity to DIB.

The DIB product may be used as such or may be hydrogenated to isooctane as described in U.S. Pat. Nos. 5,877,372 and 6,376,731. DIB and isooctane are potential fuel blending compositions.

Sulfonic acid ion exchange resins for isobutylene dimerization are typically supplied as water wet resins containing greater than 50 wt.% water. Unfortunately, the presence of water hinders the dimerization reaction and may result in detrimental unit corrosion and catalyst deactivation. Co-pending U.S. application Ser. No. 11/112,502 discloses a process for producing diisobutylene from isobutylene. The process comprises first forming dry sulfonic acid resin by contacting water wet sulfonic acid resin catalyst with a first reaction feed comprising isobutylene under conditions effective to produce tertiary butyl alcohol from the reaction of isobutylene and water, and then contacting the dry sulfonic acid resin with a second reaction feed comprising isobutylene under conditions effective to dimerize isobutylene to produce diisobutylene.

In sum, new methods to produce diisobutylene by dimerization of isobutylene over a sulfonic acid-type ion exchange resin catalyst are needed. Particularly needed are processes for reducing process equipment corrosion in the isobutylene dimerization process.

### SUMMARY OF THE INVENTION

This invention is a process for producing diisobutylene. The process comprises first passing water through a downflow reactor containing a bed of sulfonic acid resin to produce an effluent stream having a pH of at least 5, then dimerizing isobutylene by contacting the sulfonic acid resin with a reaction feed comprising isobutylene and tertiary butyl alcohol to produce a product stream comprising diisobutylene. The downflow reactor comprises a bottom, an inlet located above the bed of sulfonic acid resin, an outlet located below the bed of sulfonic acid resin, and inert material in the space from the bottom of the reactor to at least above the outlet.

### DETAILED DESCRIPTION OF THE INVENTION

The process of the invention comprises dimerizing isobutylene over a sulfonic acid-type ion exchange resin catalyst to produce diisobutylene. Sulfonic acid resin catalysts are well known. Commercial examples of sulfonic acid resin catalysts include Amberlyst A-15, Amberlyst A-35, Dowex 50, Duolite C20, Lewatit K2431, Purolite CT175, Purolite CT275, and the like. Sulfonic acid resin catalysts such as Amberlyst A-15 and A-35 are available in dry and water wet form.

The dimerization of isobutylene using sulfonic acid resin catalysts is well known in the art and has been described in U.S. Pat. Nos. 4,100,220, 4,447,668, 5,877,372, and 6,376,731, the teachings of which are hereby incorporated by reference.

The process of the invention utilizes a downflow reactor. Downflow reactors are well known in the art. The downflow reactor of the invention contains a bed of the sulfonic acid resin catalyst. The downflow reactor also comprises a bottom, an inlet located above the bed of sulfonic acid resin, an outlet located below the bed of sulfonic acid resin, and inert material in the space from the bottom of the reactor to at least above the outlet. Preferably, the downflow reactor also has a drain at the bottom of the reactor, to allow for the removal of any undesired phases that may accumulate at the bottom of the reactor below the reactor outlet.

The inert material is preferably large solid particles of a particular shape. Preferably, the inert materials are spherical but may also be other shapes such as raschig rings, berl saddles, or extrudates (cylinders). Any other conventional inert material shapes may be employed. Spheres and extrudates are especially preferred. The inert material particles are preferably larger in dimension than the sulfonic acid resin catalyst particles. The inert materials are composed of substances that are inert to the dimerization of isobutylene. Preferably, the inert materials can be alumina, metals (such as aluminum or steel), glass, ceramic, stoneware, lundum, or mixtures thereof. The inert materials act to fill any reactor dead space below the reactor outlet, and aid in the prevention of catalyst infiltration into the dead space.

The inventors have found that corrosion of the reactor or equipment downstream of the downflow reactor may result by operation of isobutylene dimerization. The sulfonic acid resins typically have residual acidity. In addition, water may be produced by the dehydration of co-fed TBA at higher reaction temperatures. The produced water may desulfonate the sulfonic acid resin catalyst and may result in an acidic aqueous phase within the process.

The process of the invention thus comprises first passing water through the downflow reactor to contact the bed of sulfonic acid resin to produce an effluent stream having a pH of at least 5. This step is useful for reducing the residual acidity of the sulfonic acid resin. Preferably, the water is contacted with the resin at a temperature of 20° C. to 100° C. and at a pressure of from 0 to 1000 psig. The water that is used in the contacting step is preferably significantly free of impurities. By “significantly free,” it is meant that the water contains less than 10,000 ppm impurities (preferably less than 2000 ppm) and has a neutral pH in the range of 6 to 8.

It is preferred to pass the contacting water through the resin as a flowing stream such that water effluent is continually carried away from the fixed bed. Liquid hourly space velocities in the range of from 0.1 to 24 are generally satisfactory. The water contacting step is performed until the pH of the effluent stream is measured at a pH of at least 5.

Optionally, the resin may be contacted with an inert gas after the water wash step in order to remove excess water from the resin. In this optional inert gas contact step, the resin may be contacted with an inert gas at a temperature of from 20° C. to 100° C. The inert gas is preferably substantially free of oxygen (i.e., less than 10,000 ppm mole oxygen) and is preferably nitrogen, helium, argon, neon, carbon dioxide, and the like.

The sulfonic acid resin may also be optionally contacted with at least one bed volume of a wash stream in order to remove a majority of water from the resin catalyst. Preferably, the sulfonic acid resin is contacted with at least two bed volumes of a wash stream. Preferably, the wash stream comprises tertiary butyl alcohol, and is at least 60 percent tertiary butyl alcohol by weight, but most preferably contains greater than 99 percent tertiary butyl alcohol by weight.

If utilized, the tertiary butyl alcohol contact step is preferably performed at a temperature of from 20° C. to 100° C. and at a pressure of from 0 to 1000 psig, and is carried out in a continuous manner such that the wash stream is passed through the resin as a flowing stream and the effluent stream is continually carried away from the resin. If performed in a continuous manner, liquid hourly space velocities in the range of from 0.1 to 10 are generally satisfactory.

Most preferably, the excess water is drained from the bottom of the reactor after the effluent stream has reached a pH of at least 5, and then the sulfonic acid resin catalyst is utilized in olefin dimerization immediately.

Following the water contact step, the sulfonic acid resin catalyst is used in the isobutylene dimerization reaction. The dimerization step comprises contacting the sulfonic acid resin with a reaction feed comprising isobutylene and tertiary butyl alcohol to produce a product stream comprising diisobutylene.

The reaction feed may include any source of isobutylene, including Cat B-B (sometimes known as Refinery B-B), raffinate streams, and isobutylene produced by the dehydration of tertiary butyl alcohol as described in U.S. Pat. Nos. 5,625,109, 3,510,538, 4,165,343, and 4,155,945. Preferably, the isobutylene is produced by the dehydration of tertiary butyl alcohol. The production of tertiary butyl alcohol by means of the Oxirane process is well known and widely practiced on an industrial scale. See, for example, U.S. Pat. No. 3,351,635. Tertiary butyl alcohol is contained in the first reaction feed as a selectivity enhancing modifier for isobutylene dimerization. The use of tertiary butyl alcohol in isobutylene dimerization is taught in U.S. Pat. Nos. 4,100,220, 5,877,372, and 6,376,731. Preferably, the reaction feed contains at least 1 weight percent tertiary butyl alcohol, more preferably from 2 to 10 weight percent tertiary butyl alcohol, and most preferably from 3 to 8 weight percent.

The reaction feed preferably contains a diluent in addition to isobutylene and tertiary butyl alcohol. Diluents are believed to enhance dimerization selectivity by reducing isobutylene feed concentration, and to aid in removal of the reaction exotherm. Preferably, the diluent is a C₃-C₁₀ hydrocarbon, more preferably a C₈ hydrocarbon in particular isooctane or diisobutylene. Most preferably, the diluent is diisobutylene. The use of alkane diluents in isobutylene dimerization is taught in U.S. Pat. Nos. 5,877,372 and 6,376,731. If a C₃-C₁₀ hydrocarbon diluent is used, the reaction feed will preferably contain 10 to 80 weight percent C₃-C₁₀ hydrocarbon, more preferably from 20 to 70 weight percent C₃-C₁₀ hydrocarbon, and most preferably from 30 to 60 weight percent.

Preferably, the reaction feed comprises 25 to 50 weight percent isobutylene, 3 to 8 weight percent tertiary butyl alcohol, and 30 to 60 weight percent diisobutylene.

Diisobutylene is produced by contacting the sulfonic acid resin with the reaction feed under conditions effective to dimerize isobutylene. In general, known dimerization conditions can be employed in the dimerization step. Suitable conditions include temperatures broadly in the range 50° C. to 200° C., preferably 50° C. to 150° C. Suitable pressures include those pressures sufficient to maintain the liquid phase, preferably above 50 psig (0.45 MPa), most preferably from 50 to 500 psig (0.45 to 3.55 MPa).

The dimerization product comprises diisobutylene. The dimerization product typically contains unreacted isobutylene, tertiary butyl alcohol, and water, in addition to diisobutylene. The dimerization product may also contain organic oxygenates such as acetone, methyl ethyl ketone, isobutyraldehyde, and methyl tertiary butyl ether.

The dimerization product may be utilized as is, but is preferentially purified to produce high purity diisobutylene. The diisobutylene may be purified by distillation. The purification of product stream comprising diisobutylene, isobutylene, tertiary butyl alcohol, and water is preferably performed by a two-step distillation process. First, the product stream is distilled to produce a first overhead stream comprising water and isobutylene and a first bottoms stream comprising diisobutylene and tertiary butyl alcohol. In the first distillation, all of the water is preferably taken overhead and preferably at least 98% (more preferably, at least 99.5%) of the tertiary butyl alcohol is removed in the first bottoms stream. Because the first bottoms stream is free of water, any tertiary butyl alcohol recycle stream will be free of water.

The first distillation is preferably conducted in a distillation tower wherein the top of the tower is at 80-200 psig (0.65-1.48 MPa), and more preferably at 80-85 psig (0.65-0.69 MPa), and the bottom of the tower is preferably at 85-210 psig (0.69-1.55 MPa), and more preferably at 85-90 psig (0.69-0.72 MPa). The tower overhead temperature is preferably maintained between about 40-65° C., and more preferably at 50-55° C., and the bottoms temperature is preferably maintained between about 145-205° C., and more preferably between 165-175° C. The first distillation tower preferably has at least 10 theoretical stages, more preferably at least 20 stages, with a reflux ratio (lb reflux/lb distillate) preferably of at least 0.5, and more preferably between 0.8 to 1.2.

Following the first distillation, the first bottoms stream is distilled in a second distillation tower to produce a bottoms product stream comprising diisobutylene and a second overhead stream comprising tertiary butyl alcohol and diisobutylene. If the dimerization product contains organic oxygenates, then the oxygenates typically end up in the second overhead stream.

The second distillation is preferably conducted in a distillation tower wherein the top of the tower is preferably at 40-70 psig (0.38-0.58 MPa), and more preferably at 50-60 psig (0.45-0.52 MPa) and the bottom is preferably at 50-80 psig (0.45-0.65 MPa), and more preferably 50-70 psig (0.45-0.58 MPa). The tower overhead temperature is preferably maintained between about 125-150° C., and more preferably between 135-145° C., and the bottoms temperature is preferably maintained between about 160-195° C., and more preferably between 170-180° C. The second distillation tower preferably has at least 10 theoretical stages, more preferably at least 20 stages, with a reflux ratio (lb reflux/lb distillate) preferably of at least 0.5, and more preferably between 0.7 to 1.1.

The first overhead stream is further processed to separate at least 30 percent of the water from the isobutylene to form an isobutylene-enriched stream. The water is separated by any known technique to remove water from a hydrocarbon stream, for instance by adsorption with adsorbents such as molecular sieves, distillation, extraction, coalescing media, or decantation. Decantation is a particularly preferred separation method. In decantation, the first overhead stream is introduced into a decanter unit where phase separation takes place. Gravity-driven phase separation of the first overhead stream results in a heavier water phase and a lighter isobutylene phase.

The separation is operated under conditions which are effective to provide an isobutylene layer in which at least 30 percent (and preferably at least 50 percent) of the water is removed, and an aqueous layer containing at most negligible amounts of isobutylene. For decantation, the volume of the decanter should be sufficient to provide a suitable residence time for phase separation to occur at a specified flow rate. The residence time for the water phase and the isobutylene phase is preferably at least 1 minute, and more preferably in the range of about 4 to 10 minutes. The pressure in the decanter should be sufficient to maintain both the isobutylene and the water in liquid phase, e.g. 50 to 150 psig (0.45-1.14 MPa) depending upon the temperature. The temperature in the decanter will preferably be between about 200 to 85° C., and more preferably between about 200 to 55° C. The solubility of water in isobutylene is less at lower temperature, but this may be expensive where refrigeration is needed.

Following separation, an isobutylene-enriched stream is produced. In decantation, for instance, the decanter overheads are recovered as an isobutylene-enriched stream in which at least 30 percent of the water has been removed, and the aqueous decanter bottoms are continuously removed from the decanter through an outlet at the bottom of the decanter. The isobutylene-enriched stream is then recycled back to the reaction zone for further dimerization reaction.

Preferably, the second overhead stream comprising tertiary butyl alcohol and diisobutylene is also recycled back to reactor. The tertiary butyl alcohol/diisobutylene mixture may be recycled immediately back to reactor or held in a tank prior to recycle. Excess tertiary butyl alcohol may also be dehydrated to isobutylene.

Overall, the process of the invention allows a significant portion of the water to be removed from any possible recycle streams so that water does not build up within the reaction process.

Following the production of diisobutylene, the diisobutylene is optionally hydrogenated to isooctane. The hydrogenation step can be carried out using conventional methods. For example, the diisobutylene may be brought into contact with hydrogen in the liquid phase at moderate temperatures and pressures. Suitable reaction temperatures vary from 0° C. to 500° C., but preferably from 25° C. to 200° C. The reaction is preferably conducted at or above atmospheric pressure. The precise pressure is not critical. Typical pressures vary from 1 atmosphere to 100 atmospheres. Any suitable hydrogenation catalyst may be used, including but not limited to Raney nickel and supported nickel, palladium, and platinum catalysts. Suitable supports for nickel, palladium, and platinum include carbon, silica, alumina, diatomaceous earth, and the like. Preferably, the hydrogenation catalyst is a supported nickel catalyst. The hydrogenation may be performed in the presence or absence of a solvent. Following hydrogenation, the isooctane product can be recovered by removing the hydrogenation catalyst and the solvent (if present) in a conventional manner, to separate isooctane.

The hydrogenation reaction may be performed using any of the conventional reactor configurations known in the art for such hydrogenation processes. Continuous as well as batch procedures may be used. For example, the catalyst may be deployed in the form of a fixed bed or slurry.

The following examples merely illustrate the invention. Those skilled in the art will recognize many variations that are within the spirit of the invention and scope of the claims.

### EXAMPLE 1

### Isobutylene Dimerization Process

Water is passed through a downflow reactor containing a bed of sulfonic acid resin. The downflow reactor has an inlet located above the bed of sulfonic acid resin, an outlet located below the bed of sulfonic acid resin, a drain at the bottom of reactor, and inert material ( 1/16 inch low surface area fused alumina spheres) in the space from the bottom of the reactor to at least above the outlet. The water is removed by the drain at the bottom of the reactor and the pH of the effluent water stream is analyzed. Once the effluent water stream has a pH of at least 5, the water flow is stopped and residual water is drained from the bottom of the reactor.

Isobutylene is then dimerized over the sulfonic acid resin catalyst in the presence of TBA (and diisobutylene from recycle, after the start of run) in accordance with the process described in U.S. Pat. No. 5,877,372. The reactor feed stream, comprising isobutylene, TBA, and water is fed to the downflow reactor. The reaction product stream, comprising diisobutylene, isobutylene, TBA, and water, is then purified by a two-step distillation procedure. The reaction product stream is passed to a first distillation tower (debutanizer). The debutanizer contains 35 ideal stages, 11 above feed and 24 below feed. The pressure is 85 psig (0.69 MPa) in the overhead and 90 psig (0.72 MPa) in the bottoms. The overhead temperature is 54° C. and the bottoms temp is 170° C. The reflux ratio is 0.9 by weight.

A debutanizer overhead stream, containing most of the unreacted isobutylene and water, is removed from the first distillation tower and is passed to a decanter operated at 47° C. The isobutylene and water are separated from one another by operation of the decanter to separate an isobutylene-enriched phase from an aqueous phase. The isobutylene-enriched phase can be recycled back to the isobutylene dimerization reactor.

The bottoms stream from the debutanizer, comprising a DIB-TBA mixture in which all of the water and most of the unreacted isobutylene is removed, is passed to a DIB distillation tower.

The DIB distillation tower contains 21 ideal stages, 9 above feed and 12 below feed. The pressure is 55 psig (0.48 MPa) in the overhead and 58 psig (0.50 MPa) in the bottoms. The overhead temperature is 141° C. and the bottoms temp is 175° C. The reflux ratio is 0.8 by weight. The bottoms stream from the DIB distillation tower contains a purified DIB stream.

The overhead stream from the DIB distillation tower comprises a DIB-TBA mixture that contains no water. This overhead stream contains most of the TBA for recycle back to the isobutylene dimerization reactor.

The flow rates of the components of the various streams (in pounds per hour) at the start of the reaction run are shown in Table 1. The flow rates of the components of the various streams (in pounds per hour) at the end of the reaction run are shown in Table 2.

This process of the invention effectively produces diisobutylene with minimal equipment corrosion due to acidity. Without the inert material in the bottom of the reactor, an acidic aqueous phase has been found to accumulate in the dead space in the bottom of the reactor that leads to damage of reactor internals and corrosion of the downstream process equipment. Without the use of water wash, acidity also builds up in the reaction system. The two-step distillation process also acts to prevent the accumulation of water within the reaction system, thereby reducing resin deactivation and corrosion.

TABLE 1
Start of Run - Component Flow Rates (lb/h)

| | | | Isobutylene | Water Phase | Overhead | Bottoms |
|---|---|---|---|---|---|---|
| | Reactor | Reactor | Phase from | from | from DIB | from DIB |
| Stream # | Feed | Effluent | Decanter | Decanter | Tower | Tower |
| Water | 307 | 200 | 127 | 73 | 0 | 0 |
| Isobutylene | 303603 | 114934 | 109631 | 0 | 1812 | 0 |
| TBA | 26559 | 26999 | 112 | 0 | 27199 | 152 |
| DIB | 33258 | 208108 | 0 | 0 | 34644 | 173464 |
| TIB | 0 | 13412 | 0 | 0 | 0 | 13412 |
| MEK | 4901 | 4901 | 23 | 0 | 4724 | 159 |
| Isobutyraldehyde | 5607 | 5607 | 208 | 0 | 5342 | 56 |
| Acetone | 1936 | 1936 | 877 | 1 | 1051 | 0 |
| Total | 386876 | 386876 | 120876 | 74 | 75000 | 187376 |

TABLE 2
End of Run - Component Flow Rates (lb/h)

| | | | Isobutylene | Water Phase | Overhead | Bottoms |
|---|---|---|---|---|---|---|
| | Reactor | Reactor | Phase from | from | from DIB | from DIB |
| Stream # | Feed | Effluent | Decanter | Decanter | Tower | Tower |
| Water | 298 | 349 | 119 | 235 | 0 | 0 |
| Isobutylene | 300999 | 112243 | 107983 | 0 | 815 | 0 |
| TBA | 15599 | 15390 | 22 | 0 | 15888 | 15 |
| DIB | 40993 | 210190 | 0 | 0 | 42730 | 167460 |
| TIB | 0 | 19645 | 0 | 0 | 0 | 19645 |
| MEK | 7803 | 7803 | 10 | 0 | 7765 | 33 |
| Isobutyraldehyde | 6453 | 6453 | 49 | 0 | 6393 | 11 |
| Acetone | 1795 | 1795 | 702 | 3 | 1087 | 0 |
| Total | 384715 | 384715 | 118765 | 238 | 75000 | 187290 |

We claim:

1. An isobutylene dimerization process, comprising:
(a) passing water through a downflow reactor containing a bed of sulfonic acid resin to produce an effluent stream having a pH of at least 5; and
(b) dimerizing isobutylene by contacting the sulfonic acid resin with a reaction feed comprising isobutylene and tertiary butyl alcohol to produce a product stream comprising diisobutylene,
wherein the downflow reactor comprises a bottom, an inlet located above the bed of sulfonic acid resin, an outlet located below the bed of sulfonic acid resin, and inert material in the space from the bottom of the reactor to at least above the outlet.

2. The process of claim 1 wherein the sulfonic acid resin is contacted with the reaction feed at a temperature of 50° C. to 200° C.

3. The process of claim 1 wherein the isobutylene is produced by dehydration of tertiary butyl alcohol.

4. The process of claim 1 wherein the reaction feed comprises at least 2 weight percent tertiary butyl alcohol.

5. The process of claim 1 wherein the reaction feed additionally comprises a C₃-C₁₀ hydrocarbon.

6. The process of claim 5 wherein the C₃-C₁₀ hydrocarbon is a C₈ hydrocarbon.

7. The process of claim 6 wherein the C₈ hydrocarbon is diisobutylene.

8. The process of claim 7 wherein the reaction feed comprises 25 to 50 weight percent isobutylene, 3 to 8 weight percent tertiary butyl alcohol, and 30 to 60 weight percent diisobutylene.

9. The process of claim 1, further comprising hydrogenating the diisobutylene to produce isooctane.

10. The process of claim 1 wherein the downflow reactor further comprises a drain at the bottom of reactor.

11. The process of claim 1 wherein the product stream comprises diisobutylene, isobutylene, tertiary butyl alcohol, and water.

12. The process of claim 11, further comprising
(c) distilling the product stream to produce a first overhead stream comprising water and isobutylene and a first bottoms stream comprising diisobutylene and tertiary butyl alcohol;
(d) separating at least 30 percent of the water from the first overhead stream to form an isobutylene-enriched stream, and recycling the isobutylene-enriched stream back to step (b); and
(e) distilling the first bottoms stream to produce a bottoms product stream comprising diisobutylene and a second overhead stream comprising tertiary butyl alcohol and diisobutylene.

13. The process of claim 12 wherein the water is separated from the first overhead stream by decantation.

14. The process of claim 12, further comprising recycling the second overhead stream back to step (b).
